(19) United States
(12) Patent Application Publication
(10) Pub. No.: US 2016/0220357 A1
(43) Pub. Date: Aug. 4, 2016

(54) NOVEL CONCEPT TO REDUCE LEFT ATRIAL PRESSURE IN SYSTOLIC AND DIASTOLIC HF PATIENTS TO TREAT PULMONARY EDEMA AND REDUCE HOSPITALIZATION RATES

(71) Applicants: Boston Scientific Scimed, Inc., Maple Grove, MN (US); Mayo Foundation for Medical Education and Research, Rochester, MN (US)

(72) Inventors: Umang Anand, Maple Grove, MN (US); Raghav Goel, Plymouth, MN (US); Roger W. McGowan, Otsego, MN (US); Daniel Ross, Watertown, MN (US); Patrick A. Haverkost, Brooklyn Center, MN (US); Mary M. Byron, Roseville, MN (US); David R. Wulfman, Minnneapolis, MN (US); James P. Rohl, Prescott, WI (US); Peter M. Pollak, Atlantic Beach, FL (US); Lyle J. Olson, Rochester, MN (US); Atta Behfar, Rochester, MN (US); Charles J. Bruce, Rochester, MN (US)

(21) Appl. No.: 15/015,298

(22) Filed: Feb. 4, 2016

Related U.S. Application Data
(60) Provisional application No. 62/111,970, filed on Feb. 4, 2015.

(51) Int. Cl. A61F 2/24

(52) U.S. Cl. CPC A61F 2/2403; A61F 2/2409; A61F 2230/0069; A61F 2210/0014

(57) ABSTRACT

Devices provided herein can include implantable transseptal flow control components adapted to be implanted in an opening in a septal wall. In a closed configuration, the implantable transseptal flow control components provided herein prevent blood from flowing through the opening. In an open configuration, the implantable transseptal flow control components provided herein allow blood to flow from the left atrium to the right atrium. In a closed configuration, implantable transseptal flow control components provided herein can be configured such that blood does not stagnate at a location proximate to a left atrium flow control component side when the pressure differential is below a second predetermined threshold pressure value.

Implantable transseptal flow control components provided herein can remain in a closed configuration when a pressure differential between the left atrium and the right atrium is less than a first non-zero predetermined threshold pressure value.

### CLAIM OF PRIORITY

This application claims the benefit of priority to U.S. Provisional Application Ser. No. 62/111,970, filed on Feb. 4, 2015, the entire contents of which are hereby incorporated by reference.

### BACKGROUND

Heart failure is a growing epidemic worldwide. In the United States, the incidence of heart failure has remained stable over the past several decades, with more than 650,000 new heart failure cases diagnosed annually. Heart failure incidence increases with age, rising from approximately 20 per 1,000 individuals aged 65 to 69 years to more than 80 per 1,000 individuals aged at least 85 years. Approximately 5,100,000 persons in the United States have clinically manifested heart failure, and the prevalence continues to rise. Patients with heart failure with reduced ejection fraction (HFrEF) or heart failure with preserved ejection fraction (HFpEF) have a poor prognosis; each of these broad types of heart failure account for about half of heart failure patients in the United States.

Shortness of breath, or dyspnea, is the symptom hallmark of heart failure due to either HFrEF or HFpEF. Dyspnea is due to pulmonary congestion, which is a consequence of elevated left atrial pressure. A subset of patients with pulmonary congestion will have pulmonary edema. Pulmonary edema is the condition where lung fluid accumulates in the air spaces and parenchyma of the lungs causing impaired ventilation, decreased gas exchange and an increased respiratory drive. The traditional treatment of pulmonary edema due to heart failure requires hospitalization and administration of intravenous diuretic therapy.

### SUMMARY

Devices, methods, and systems provided herein can reduce the left atrial pressure. In some cases, a reduction of left atrial pressure can prevent patients from going into pulmonary edema and therefore potentially improve patient outcomes, patient comfort, and reduce or eliminate hospital stays.

In some aspects, devices provided herein can include implantable transseptal flow control components. Implantable transseptal flow control components provided herein can be adapted to be implanted in an opening in a septal wall between a left atrium and a right atrium. In a closed configuration, the implantable transseptal flow control components provided herein prevent blood from flowing through the opening. In an open configuration, the implantable transseptal flow control components provided herein allows blood to flow from the left atrium to the right atrium. Implantable transseptal flow control components provided herein can remain in a closed configuration when a pressure differential between the left atrium and the right atrium is less than a first non-zero predetermined threshold pressure value. Implantable transseptal flow control components provided herein can transition into an open configuration when the pressure differential exceeds the first non-zero predetermined threshold pressure value. When in a closed configuration, implantable transseptal flow control components provided herein can be configured such that blood does not stagnate at a location proximate to a left atrium flow control component side when the pressure differential is below a second predetermined threshold pressure value. In some cases, implantable transseptal flow control components provided herein provide zero dead space when in a closed configuration below a second predetermined threshold pressure value. In some cases, implantable transseptal flow control components provided herein can be configured such that blood does not stagnate at a location proximate to either the left or right atrium flow control component sides when the pressure differential is below the second predetermined threshold pressure value. As defined herein, a pressure differential between the left atrium and the right atrium is the pressure of the left atrium in excess of the pressure of the right atrium, thus the pressure differential can be both positive (i.e, the left atrium pressure greater than the right atrium pressure) and negative (i.e., the right atrium pressure greater than the left atrium pressure. In some cases, implantable transseptal flow control components provided here will remain in a closed configuration when the pressure differential is negative.

Stagnating blood within chambers of the heart can result in thrombosis and/or blood clots around a flow control component. Normally, a flow control component is adapted to open repeatedly with each heartbeat, thus blood found in dead spaces in the flow control components' closed configurations is repeatedly flushed away. Implantable transseptal flow control components provided herein, however, are adapted to only open upon a pressure differential between the left atrium and the right atrium exceeding a predetermined threshold value, thus implantable transseptal flow control components provided herein may not open for hours, days, weeks, or even months at a time. Accordingly, implantable transseptal flow control components provided here allow for the reduction of or limiting of a pressure difference between the left and right atrium that also mitigates issues associated with stagnating blood.

In some aspects, an implantable transseptal flow control component provided herein is adapted to be implanted in an opening in a septal wall between a left atrium and a right atrium and adapted to prevent blood from flowing through the opening when in a closed configuration. The flow control component can be adapted to remain in a closed configuration when a pressure differential between the left atrium and the right atrium is less than a first non-zero predetermined threshold pressure value and transition into an open configuration when the pressure differential exceeds the first non-zero predetermined threshold pressure value. The flow control component can be configured such that blood does not stagnate at a location proximate to a left atrium flow control component side when the pressure differential is below a second predetermined threshold pressure value. In some cases, the flow control component can be configured such that a periodic opening of the flow control component during each cardiac cycle is less than 100 ml/minute to prevent stagnation. The implantable flow control component provided herein can include at least a first member. In some cases, the second predetermined threshold pressure value is less than or equal to the first non-zero predetermined threshold pressure value. In some cases, the first predetermined threshold pressure value is between 10 mmHg and 15 mmHg.

In some cases, the open configuration defines a passage through the flow control component that increases with an increasing pressure differential after the pressure differential exceeds the first non-zero predetermined threshold pressure value. In some cases, the size of the opening can increase in diameter in a step-like function relative to the pressure differential. In some cases, the size of the opening can increase in diameter exponentially over a desired pressure range. In some cases, the opening can increase in diameter linearly over a desired pressure range.

In some cases, the first member is compliant. In some cases, the first member can be adapted to flex in response to pressure differential. In some cases, the first member defines a collapsed passage there through when the pressure differential is less than the second predetermined threshold pressure value.

In some cases, the flow control component can include a second member. In some cases, the second member configured to form a shape-stable support structure when the flow control component is implanted. The term “shape-stable” as used herein means that it is less compliant than the first member. In some cases, the shape-stable second member can be adapted to expand from a retracted configuration to an expanded configuration that is less compliant than the first member. In some cases, the shape-stable member can include compliant materials that interlock when in an expanded configuration to be less compliant than the first member. In some cases, the shape-stable member comprises inelastic materials.

In some cases, the second member defines passage there through and the first member overlies and seals the passage when the pressure differential is below the second predetermined threshold pressure value. In some cases, the first member has a semi-circular shape. In some cases, the first member defines at least one passage there through. In some cases, the first and second members are both disk shaped and connected along a periphery of the disks or at a central location of each disk. In some cases, the first member comprises a shape memory metal. In some cases, the first member forms at least one lobe structure.

In some cases, the flow control component comprises a spring, a magnet, or a combination thereof.

In some cases, the flow control component can include a controller adapted to detect the pressure differential and control the opening and closing of the flow control component based on the detected pressure differential.

In some aspects, an implantable transseptal flow control component provided herein can include a shape-stable member and a compliant member. The flow control component can be adapted to be implanted in an opening in a septal wall between a left atrium and a right atrium. The shape-stable member and the compliant member can define a passage there through. The shape-stable member and the compliant member can be attached at at least one location and overlying each other to seal off any passages through the flow control component when the flow control component is in a closed configuration to prevent blood from flowing through the opening. The flow control component can be adapted to remain in a closed configuration when a pressure differential between the left atrium and the right atrium is less than a first non-zero predetermined threshold pressure value and transition into an open configuration when the pressure differential exceeds the first non-zero predetermined threshold pressure value. In some cases, the shape-stable member can be adapted to be collapsed for insertion and expanded for placement within the opening, the shape-stable member being compliant in the expanded configuration. In some cases, the shape-stable member and the compliant member are each disk shaped and attached and sealed together at a central location or along a periphery of at least one disk. In some cases, the compliant member can include a shape memory wire therein.

In some aspects, an implantable transseptal flow control component provided herein can include compliant member defining a collapsed passage there through. The compliant member can be adapted to be implanted in an opening in a septal wall between a left atrium and a right atrium. The collapsed passage can be adapted to remain in a closed configuration when a pressure differential between the left atrium and the right atrium is less than a first non-zero predetermined threshold pressure value and transition into an open configuration when the pressure differential exceeds the first non-zero predetermined threshold pressure value.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1D depict views of a heart showing the placement of a first embodiment of an implantable transseptal flow control component provided herein. FIGS. 1A and 1B depict the placement of the implantable transseptal flow control component from the right atrium. FIGS. 1C and 1D depict the placement of the implantable transseptal flow control component from the left atrium.

FIGS. 2A-2F depict how the first embodiment shown in FIGS. 1A-1D can alternate between stages of open and closed configurations when implanted.

FIGS. 3A-3G depict how an implantable transseptal flow control component according to a second embodiment can alternate between stages of open and closed configurations when implanted.

FIGS. 4A and 4B depict an implantable transseptal flow control component according to a third embodiment.

FIGS. 5A and 5B depict an implantable transseptal flow control component according to a fourth embodiment.

FIGS. 6A and 6B depict an implantable transseptal flow control component according to a fifth embodiment.

FIGS. 7A-7F depict an implantable transseptal flow control component according to a sixth embodiment. FIGS. 7A-7C depict the component in a closed configuration. FIGS. 7D-7F depict the component in an open configuration.

FIGS. 8A-8C depict an implantable transseptal flow control component according to a seventh embodiment.

FIGS. 9A-9E depict an implantable transseptal flow control component according to an eighth embodiment. FIGS. 9A-9D depict the flow control component in a closed configuration. FIG. 9E depicts the flow control component in an open configuration.

FIG. 10 depict an implantable transseptal flow control component according to a ninth embodiment.

FIGS. 11A-11I depict an implantable transseptal flow control component according to a tenth embodiment.

FIGS. 12A and 12B depict an implantable transseptal flow control component according to an eleventh embodiment.

FIGS. 13A-13D depict an implantable transseptal flow control component according to a twelfth embodiment.

FIG. 14 depict an implantable transseptal flow control component according to a thirteenth embodiment.

FIG. 15 depict an implantable transseptal flow control component according to a fourteenth embodiment.

FIG. 16 depict an implantable transseptal flow control component according to a fifteenth embodiment.

FIG. 17 depicts possible iris configurations of an implantable transseptal flow control component.

FIGS. 18A-18C depict a support frame for a collapsible compliant member, which can be used with implantable transseptal flow control components provided herein.

FIGS. 19A-19C depicts an example of a delivery catheter, which can be used with implantable transseptal flow control components provided herein. FIGS. 19B and 19C depict how the flow control component provided herein can be loaded into a delivery catheter.

Like reference symbols in the various drawings indicate like elements.

### DETAILED DESCRIPTION

Implantable transseptal flow control components provided herein are designed to be placed in an opening in the septum between the left atrium and the right atrium, to open once a non-zero predetermined pressure difference between the left atrium and the right atrium is reached, and to include a structure within the left atrium such that blood does not stagnate around the flow control component in at least the left atrium. Method provided herein include methods of making implantable transseptal flow control components and methods of implanting implantable transseptal flow control components in an opening in the septum between the left atrium and the right atrium. Systems provided herein can include an implantable transseptal flow control component and a delivery catheter.

Although a variety of different embodiments of implantable transseptal flow control components are provided herein, each can limit the stagnation of blood in and around the flow control component, particularly in the left atrium. In some cases, flow control components provided herein provide zero dead space when in a closed configuration below a second predetermined threshold pressure value. In some cases, implantable transseptal flow control components provided herein can be configured such that blood does not stagnate at a location proximate to either the left or right atrium flow control component sides when the pressure differential is below the second predetermined threshold pressure value. Stagnating blood within chambers of the heart can result in thrombosis and/or blood clots around a flow control component. Normally, a flow control component is adapted to open repeatedly with each heartbeat, thus blood found in dead spaces in the flow control components' closed configurations is repeatedly flushed away. Implantable transseptal flow control components provided herein, however, are adapted to only open upon a pressure differential between the left atrium and the right atrium exceeding a predetermined threshold value, thus implantable transseptal flow control components provided herein may not open for hours, days, weeks, or even months at a time. Accordingly, implantable transseptal flow control components provided here allow for the easing of a pressure difference between the left and right atrium without disallowing for any pressure differential and limiting issues associated with stagnating blood. In some cases, implantable transseptal flow control components provided herein can be adapted to fluctuate between a closed configuration and a partially open configuration for normal healthy pressure conditions within the left and right atriums.

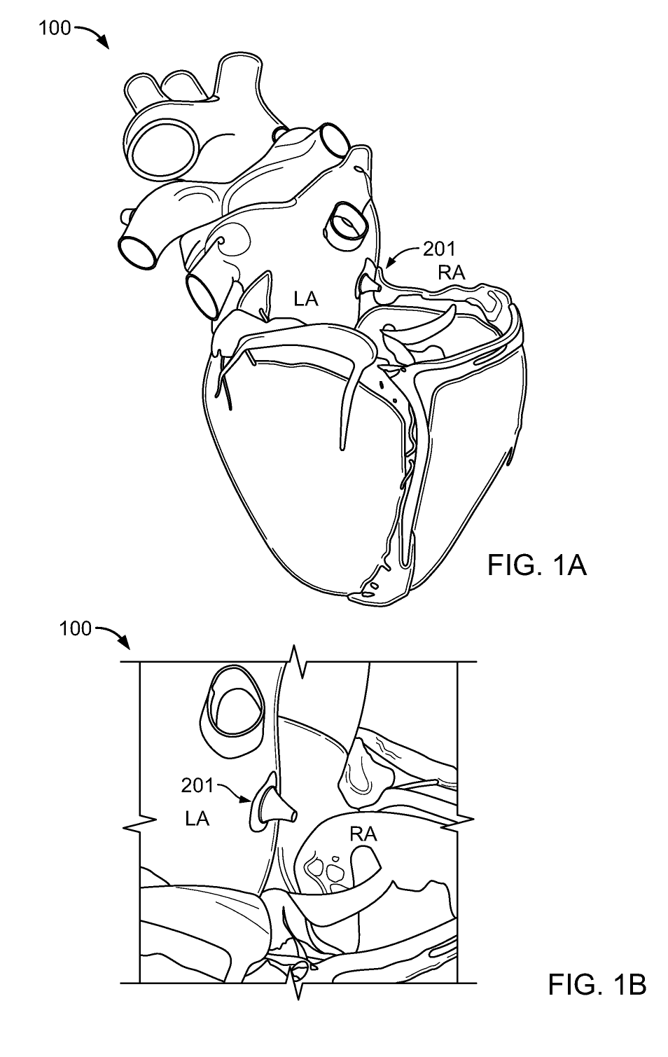

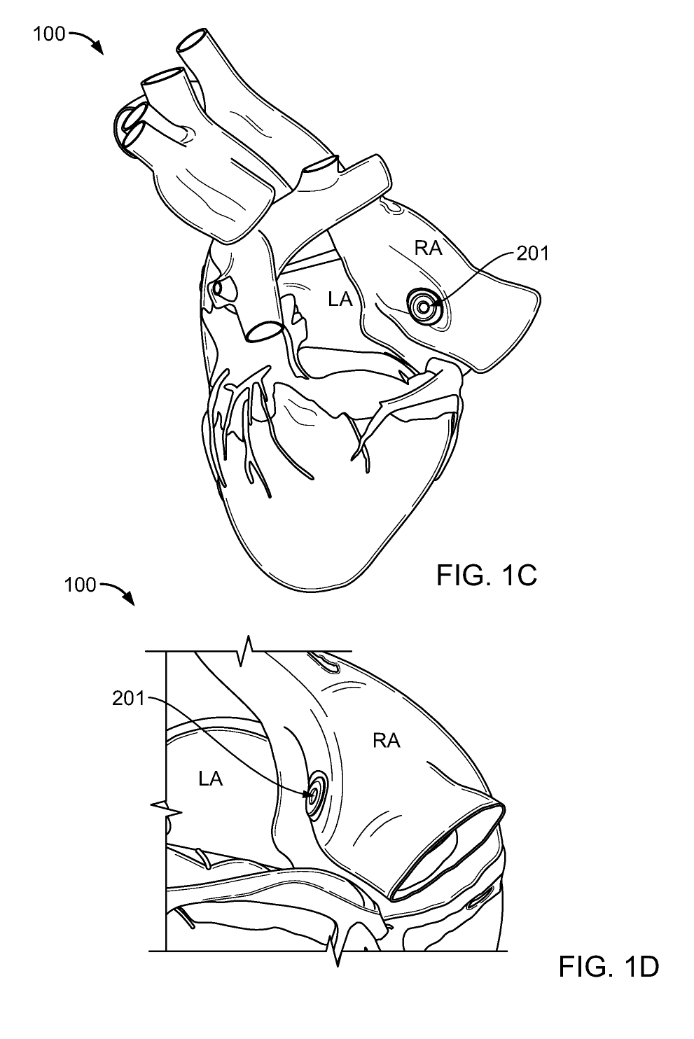

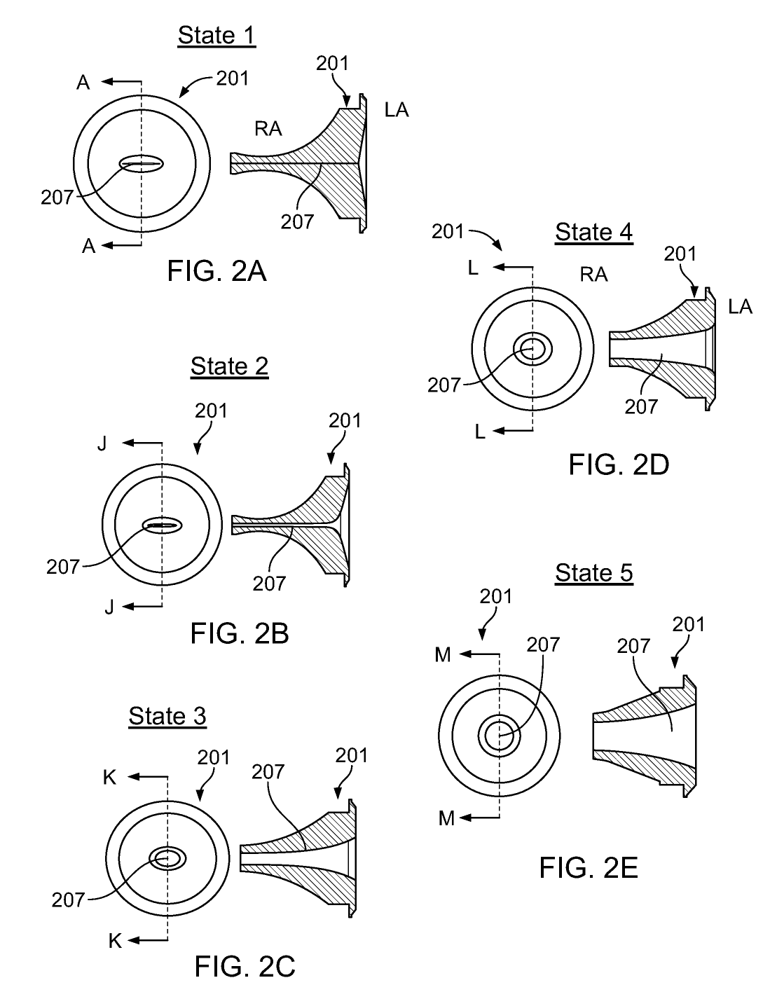

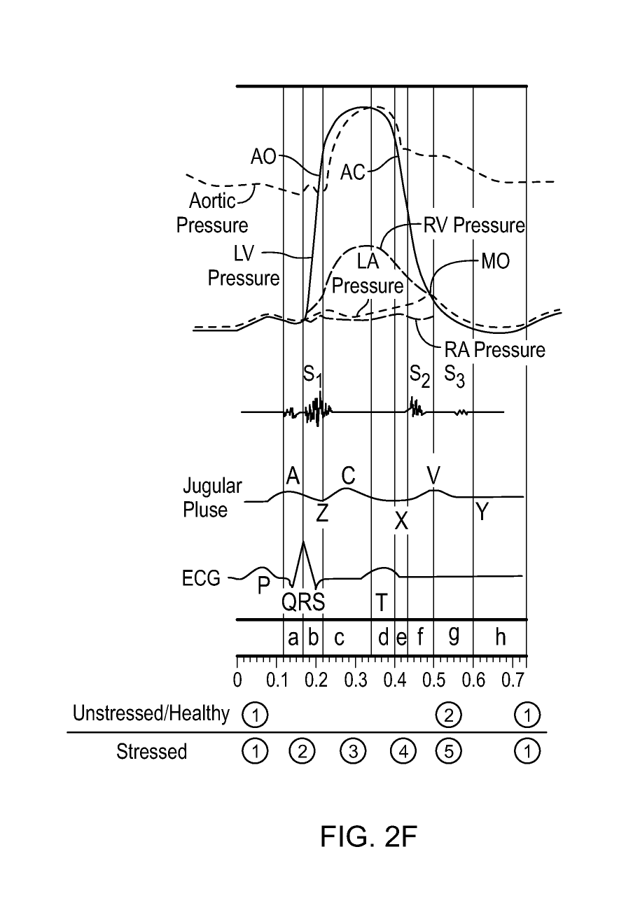

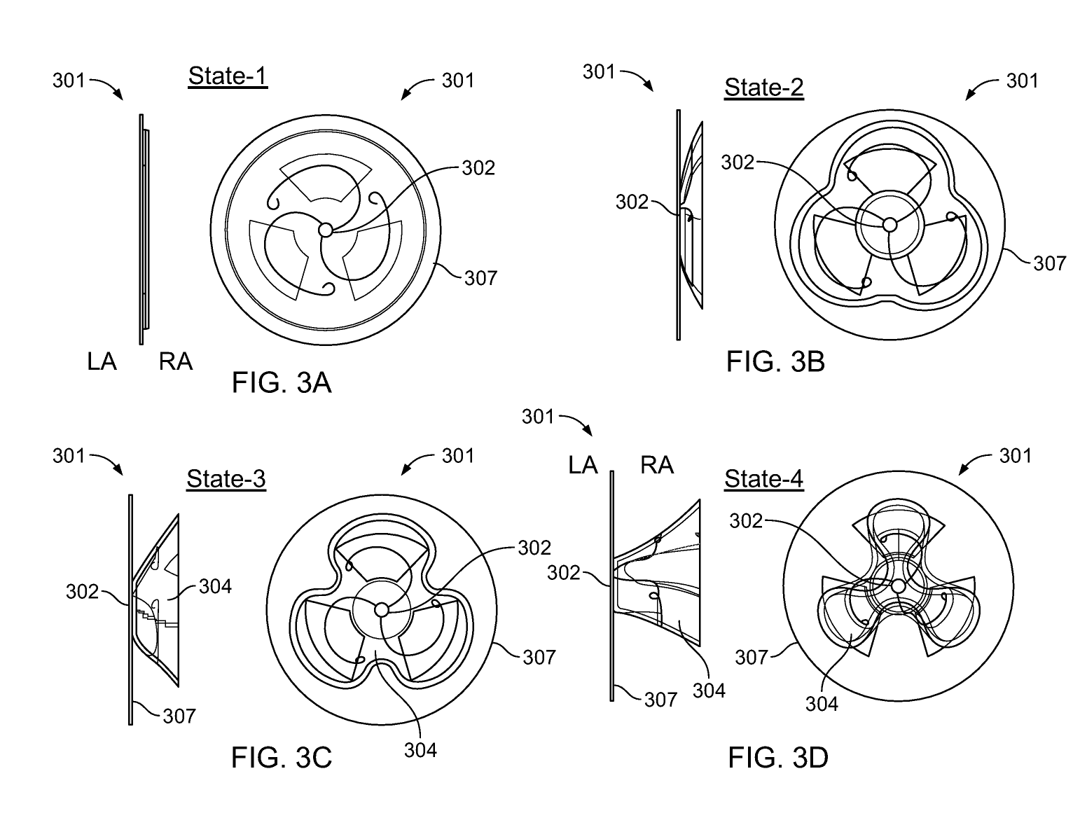

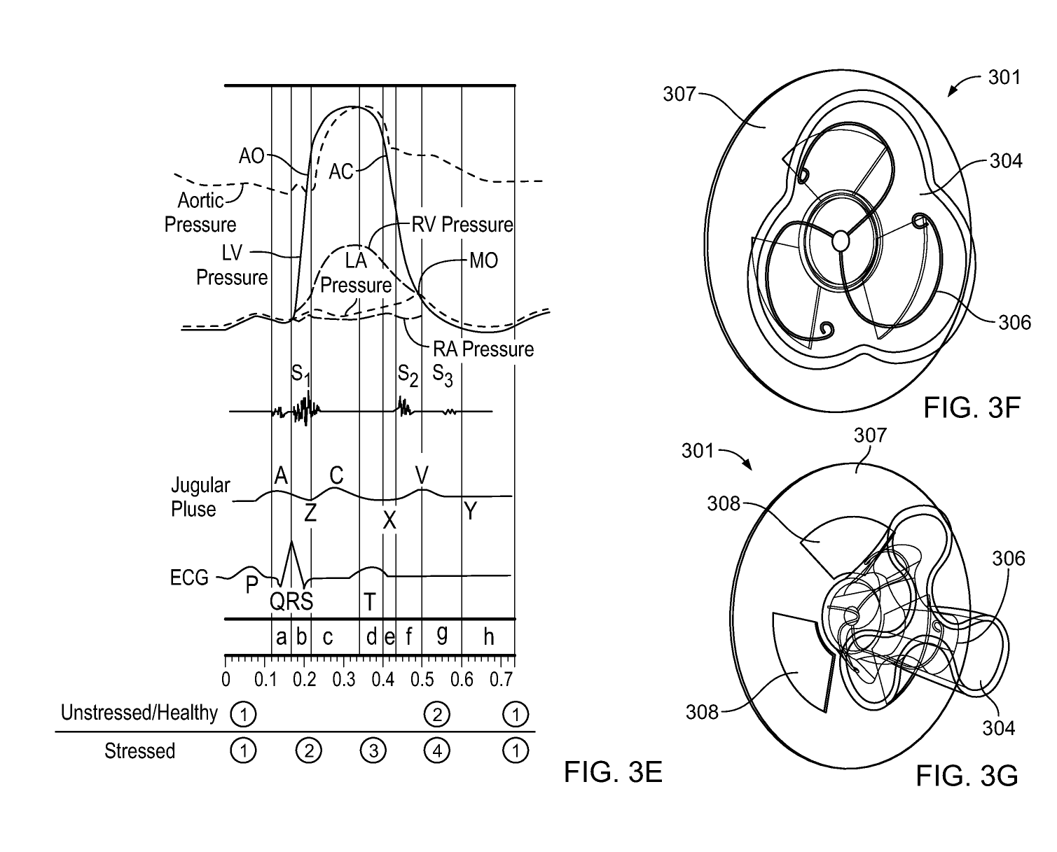

FIGS. 1A-1D depict views of a heart 100 showing the placement of a particular embodiment of an implantable transseptal flow control component 201 provided herein. As shown in FIGS. 1A and 1B, implantable transseptal flow control component 201 has a duckbill (also described as a bill) that extends into the right atrium (RA). As shown in FIGS. 1C and 1D, implantable transseptal flow control component 201 can be approximately flush with the septum in the left atrium (LA) to limit the stagnation of blood around flow control component 201 in the left atrium. In some cases, flow control component 201 can be inserted into an opening formed at the location of the Fossa Ovalis. In some cases, methods provided herein can include cutting an opening in the septum between the left atrium and the right atrium. In some cases, the hole can be cut by a delivery catheter. In some cases, systems provided herein can include a delivery device adapted to first cut an opening in the septum and then deliver a flow control component (e.g., flow control component 201) into the opening. In some cases, a delivery device provided herein is adapted to be positioned in the left atrium or the right atrium transvascularly. Although FIGS. 1A-1D depict the placement of flow control component 201, which is shown in greater detail in FIGS. 2A-2E and discussed below in greater detail below, other embodiments of implantable transseptal flow control components provided herein, such as those shown in FIGS. 3A-16, can be placed in an opening in the same location.

Flow control components, such as flow control component 201 of FIGS. 1A-1D, can include a circular cross-sectional body portion suitably shaped for insertion into the opening formed at the Fossa Ovalis. In some cases, the circular cross-sectional body portion has a diameter of about 0.5 inches (about 12 millimeters (mm)). In some cases, the diameter of the circular cross-sectional body portion is between 0.4 inches and 0.6 inches (between 10 mm and 15 mm). Other embodiments of the flow control component discussed herein can have the same or substantially the same diameter.

FIGS. 2A-2E better depict the embodiment shown in FIGS. 1A-1D and shows how it can alternate between stages of open and closed configurations when implanted. Each of FIGS. 2A-2E depicts a frontal view (e.g., from the right atrium) and a cross-sectional view. FIG. 2F depicts how the stages of FIGS. 2A-2E correspond to heart chamber pressures.

As shown in FIGS. 2A-2E, the structure of flow control component 201 can be contoured to close with increasing RA pressure. In some cases, such as that shown, flow control component 201 includes a bill adapted to extend into a right atrium (RA) space. Flow control component 201 can include an open lip-like geometry presented to the LA space. Flow control component 201 can have a smooth LA face, which can limit sites for turbulent eddy generation. The LA face can be geometrically biased to open with increasing LA pressure relative to the right atrium.

Flow control component 201 defines a passage 207 therethrough. In some cases, passage 207 can be defined as an opening having a cross-sectional dimension, e.g., a diameter, of between 5 to 10 millimeters. Passage 207 is collapsed when there is no pressure differential between opposite sides of flow control component 201, as shown in FIG. 2A. Alternatively, as shown in FIGS. 2B-2E, flow control component 201, can open when the pressure differential between the pressure in the left atrium and the pressure in the right atrium exceeds a predetermined threshold pressure value, e.g., the first predetermined threshold pressure. In some cases, the predetermined threshold pressure value is about 10 kilopascals (kPa) (about 7.5 millimeters of mercury (mmHg)). Accordingly, in some cases, passage 207 can open when the pressure differential between the pressure in the left atrium and the pressure in the right atrium exceeds about 10 kPa (about 7.5 mmHg). In some cases, the predetermined threshold pressure value is a pressure value between 0.7 kPa and 1.3 kPa (between 7.5 mmHg and 10 mmHg), or between 1.3 kPa and 2 kPa (between 10 mmHg and 15 mmHg).

Flow control component 201 can include an elastic material. In some cases, flow control component 201 have a double walled tubular structure. In some cases, a space between walls can be filled with an elastic material. Suitable materials for the walls can include elastomeric polymers such as silicones, styrene-isobutylene-styrenes (SIBS), poly-isobutylene polyurethanes (PIB-PUR), biocompatible fluoropolymers, para-methoxy-N-methylamphetamines (PMMA), silicones, polyethylene terephthalates (PET), polytetrafluoroethylenes (PTFE), and combinations thereof. Suitable materials for material included in a space between the walls include polymer foams, braided mesh made of one or a combination of metal and synthetic polymer materials such as nitinol (NiTi), polyurethanes, silicones, biocompatible fluoropolymers, poly(styrene-block-isobutylene-block-styrene) (SIBS), para-methoxy-N-methylamphetamines (PMMA), silicones, polyethylene terephthalates (PET), polytetrafluoroethylenes (PTFE), and combinations thereof.

FIG. 2F depicts the pressures of different heart chambers for both an exemplary healthy/nondiseased patient and for an exemplary diseased patient. As shown, the health/nondiseased patient shows a slightly higher pressure in the left atrium than in the right atrium. A diseased patient during periods of no exercise and no stress having a flow control component 201 would have the flow control component 201 mostly remain in state 1 (FIG. 2A), where it is closed, but periodically have a pressure differentially increase sufficiently to partially open valve 201 in state 2 (FIG. 2B). In state 1, flow control component 201 is fully closed, and occurs at points in cardiac cycle when the RA pressure is equal to or greater than the LA pressure or when the pressure differential between the left atrium and the right atrium is less than a predetermined threshold value, which can be less than the maximum pressure differential experienced in a healthy/nondiseased heart. State 1 can prevent regurgitation, but a periodic state 2 can provide a clearing function for removing residual blood from a coaptation zone in passage 207 to prevent adhesion between opposite sides of a collapsed passage 207. As shown, state 2 has passage 207 only partially open. In some cases, state 2 can be configured to prevent more than about 2% of the cardiac output from passing through flow control component 201 during a cardiac cycle (e.g., about 50 ml/minute of blood).

In some cases, flow control component 201 can be configured such that blood does not stagnate at a location proximate to a left atrium flow control component side when the pressure differential is below a second predetermined threshold pressure value. In some cases, the flow control component 201 can be configured such a periodic opening of the flow control component during each cardiac cycle is less than 100 ml/minute to prevent stagnation.

For a patient in a diseased state with or without exercise, LA pressure can be significantly elevated above normal. As shown in FIGS. 2C-2E, larger pressure differences between left atrium and right atrium can force open the flow control component orifice to an increasingly larger diameter. Flow control component 201 changes states 1-5 depending on pressure difference in cardiac cycle. As will be discussed below, other embodiments of flow control components provided herein can also provide for increasing passage sizes with increasing pressure differentials.

FIGS. 3A-3G depict how an implantable transseptal flow control component 301 according to a second embodiment can alternate between stages of open and closed configurations when implanted. Flow control component 301 includes a shape-stable disk 307 and a compliant disk 304 connected centrally 302. Shape-stable disk 307 can be flexible so that it can be collapsed and expanded between a non-deployed state (for delivery) and an expanded deployed state. When in the deployed state, shape-stable disk 307 is strong enough to resist deformation due to the blood flow through and/or around shape-stable disk 307. Shape-stable disk 307 defines passages 308 therethrough. As will be discussed below, shape-stable disk 307 can be collapsible (e.g., for intravascular delivery). In some cases, for example, shape-stable disk 307 can include a collapsible frame and a non-elastic but flexible sheet.

In some cases, flow control components can be fully or partially contained within the inter-atrial septum. In some cases, flow control components can be fully or partially project outwardly from one or both sides of the septum. In some cases, at least a portion of the flow control component, e.g. shape-stable disk 307 of FIGS. 3A-3G or a central portion of flow control component, can have a thickness of about 1 mm to about 5 mm. In some cases, portions of the flow control components have a thickness of about 1 to about 2 mm, or about 2 mm to about 5 mm, or about 5 mm to about 10 mm.

Compliant disk 304 can include a shape memory wire 306 embedded in the compliant member to urge the compliant disk 304 towards shape-stable disk 307. As shown in FIG. 3A, state 1 depicts a closed configuration where compliant disk 304 overlies and seals passages 308 due to a lack of a pressure differential exceeding a predetermined threshold value. Shape memory wires 306 can be designed to control a pressure differential required to overcome the shape memory properties of the shape memory wires 306. In some cases, shape memory materials discussed herein can be a nickel-titanium alloy (e.g., nitinol). As shown in FIG. 3B, state 2 results in a flexing of compliant disk 304 to allow flow of blood through passages 308. Similar to that discussed above with regards to FIGS. 2A-2F, a diseased patient during a period of no exercise and no stress can have heart pressures that cause flow control component 301 to alternate between state 1 and state 2, optionally with each cardiac cycle, to periodically flush residual blood from coaptation zone between compliant disk 304 and shape-stable disk 307. As shown in FIGS. 3C-3E, a diseased heart can experience higher pressure differentials and thus reach state 3 and state 4. FIGS. 3F and 3G depict perspective views of flow control component 301 in state 2 and state 4, respectively.

In some cases, flow control component 301 can be configured such that blood does not stagnate at a location proximate to a left atrium flow control component side when the pressure differential is below a second predetermined threshold pressure value. In some cases, the flow control component 301 can be configured such a periodic opening of the flow control component during each cardiac cycle is less than 100 ml/minute to prevent stagnation.

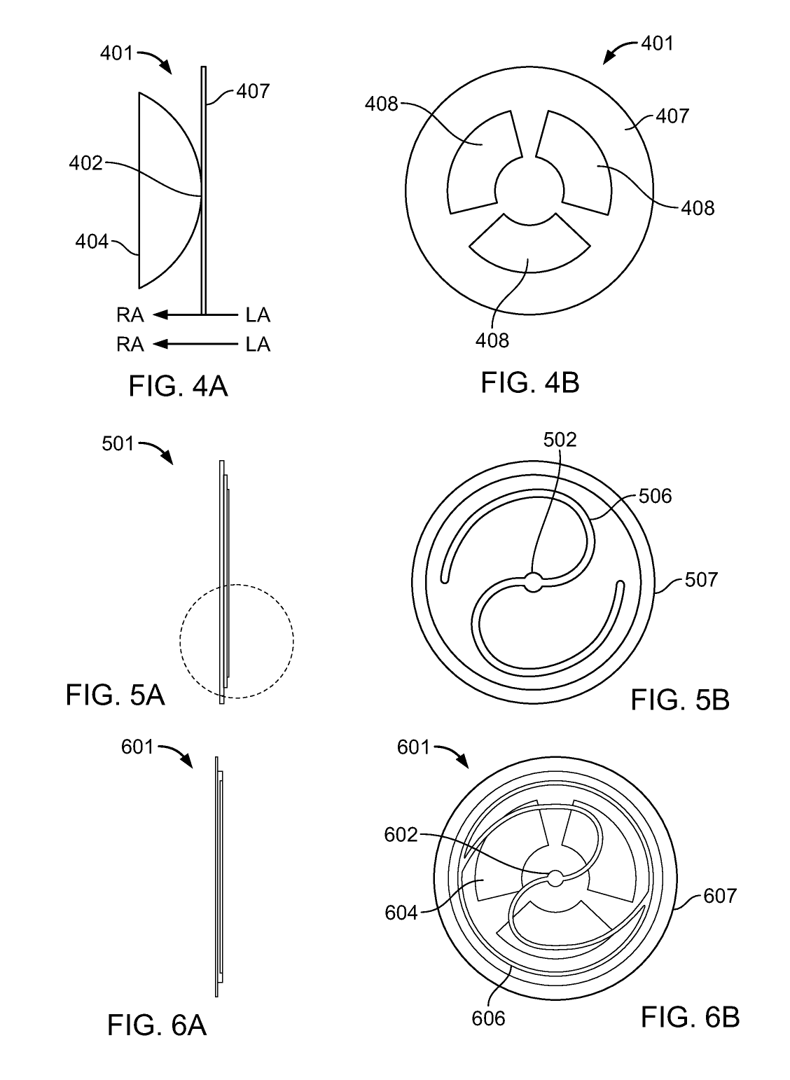

FIGS. 4A and 4B depict an implantable transseptal flow control component 401 according to a third embodiment. FIGS. 5A and 5B depict an implantable transseptal flow control component according to a fourth embodiment. FIGS. 6A and 6B depict an implantable transseptal flow control component according to a fifth embodiment. Each of FIGS. 4A-6B depict embodiments that are similar to that depicted in FIGS. 3A-3G, but differ with regard to the compliant disk. As shown in FIGS. 4A and 4B, flow control component 401 includes a compliant disk 404 secured to a shape-stable disk 407 at a central axis 402. Flow control component 401 is shown in a state when a pressure differential is greater than a predetermined threshold value such that blood can flow through passages 408 in shape-stable disk 407. As shown, FIGS. 4A and 4B do not include a shape memory (e.g., nitinol) wire. As shown in FIGS. 5A and 5B, flow control component 501 includes a compliant disk 504 secured to a shape-stable disk 507 at a central axis 502. Flow control component 501 is shown in a state when a pressure differential is less than a predetermined threshold value such that the flow control component is closed and blood cannot flow through passages 508 in shape-stable disk 507. As shown, FIGS. 5A and 5B have a compliant disk including a shape memory (e.g., nitinol) wire. As shown in FIGS. 6A and 6B, flow control component 601 includes a compliant disk 604 secured to a shape-stable disk 607 at a central axis 602. Flow control component 601 is shown in a state when a pressure differential is less than a predetermined threshold value such that the flow control component is closed and blood cannot flow through passages 608 in shape-stable disk 607. As shown, FIGS. 6A and 6B have a compliant disk including a shape memory (e.g., nitinol) wire.

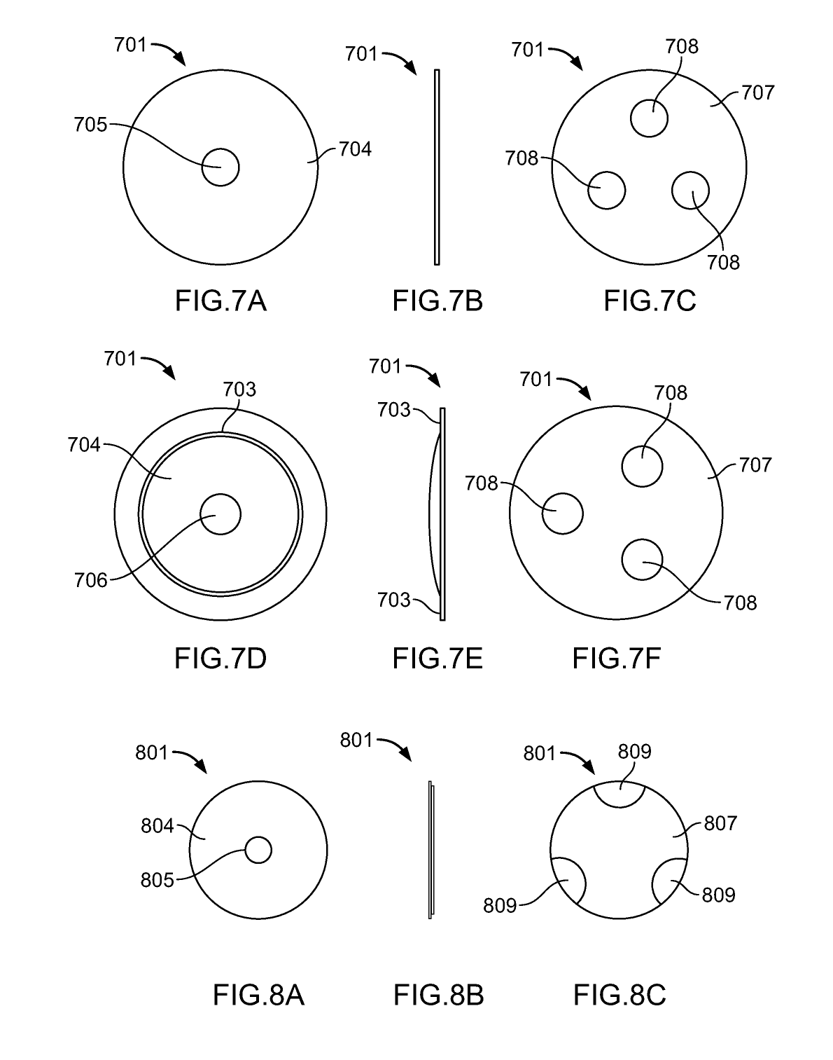

FIGS. 7A-7F depict an implantable transseptal flow control component 701 according to a sixth embodiment. FIGS. 7A-7C depict it in a closed configuration, e.g., state 1. FIGS. 7D-7F depict it in an open configuration, e.g., state 3. FIGS. 7A and 7D depict views from the right atrium. FIGS. 7C and 7F depict views from the left atrium. FIGS. 7B and 7E depict side views. As shown in FIGS. 7A-7F, compliant disk 704 is sealed to shape-stable disk 707 along a periphery of each disk. Shape-stable disk 707 defines at least one passage 708 there through and compliant disk 704 defines at least one passage 705 there through, but passages 705 and 708 are non-aligned such that compliant disk 704 overlies the holes in shape-stable disk 707 when the pressure differential is below a predetermined threshold. As shown in FIG. 7E, a pressure differential above a predetermined threshold can cause compliant disk 704 to balloon out to form a path for fluid to flow between passage 705 and passage 708.

FIGS. 8A-8C depict an implantable transseptal flow control component 801 according to a seventh embodiment. As shown a compliant disk 804 defines a central passage 805 there through and shape-stable disk 807 includes peripheral cutouts 809. Compliant disk 804 and shape-stable disk 807 can be secured together intermittently along a periphery of the disk to allow for a space to open between the compliant disk 804 and the shape-stable disk 807 along the periphery (e.g., at the peripheral cutouts 809). FIGS. 8A-8C depict flow control component 801 in a closed configuration (state 1), but open configurations (states 2-4) can also exist with increasing pressure differences.

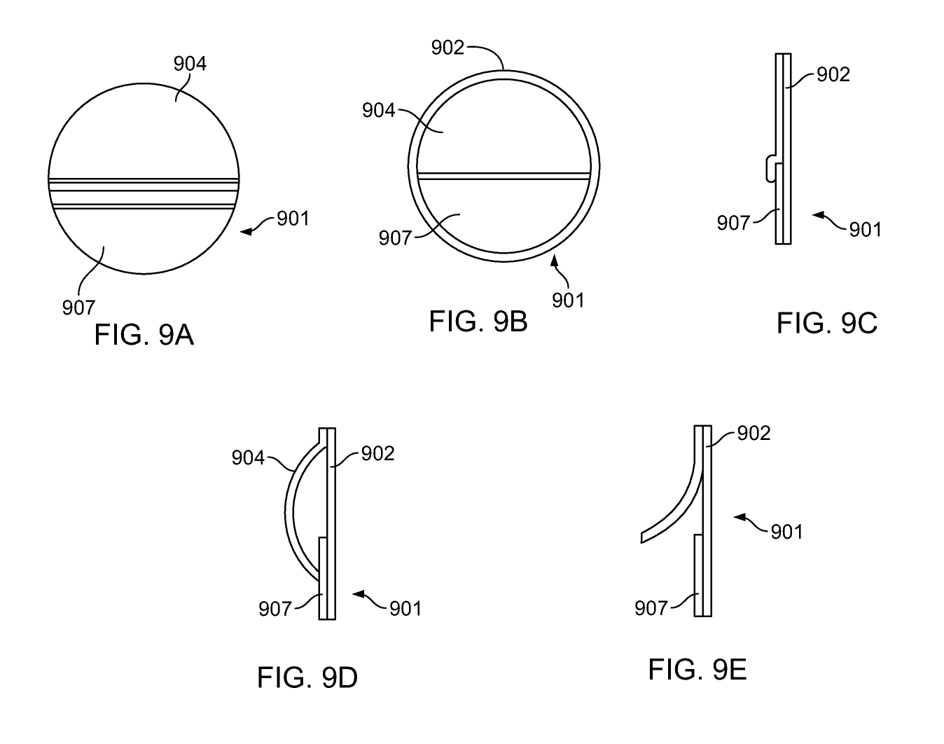

FIGS. 9A-9E depict an implantable transseptal flow control component 901 according to an eighth embodiment. As shown, flow control component 901 includes a shape-stable ring 902, a shape-stable semicircle member 907 secured to a portion of shape-stable ring 902, and a compliant flap 904 secured to the shape-stable ring 902 such that flap 904 and shape-stable semicircle member 907 can provide a closed configuration, such as shown in FIGS. 9A-9C. FIG. 9A depicts a view from the RA side. FIG. 9B depicts a view from the LA side. FIG. 9C depicts a side view, showing a closed position. FIG. 9D depicts a side view showing a partial ballooning of flexible flap 904 while flow control component 901 remains closed. In some cases, this ballooning can pulsate during unstressed cardiac cycles to provide a clearing function to clear residual blood from areas along the interface between flap 904 and shape-stable semicircle member 907. FIG. 9E depicts flow control component 901 in an open configuration after a pressure differential between left atrium and right atrium exceeds a predetermined threshold value.

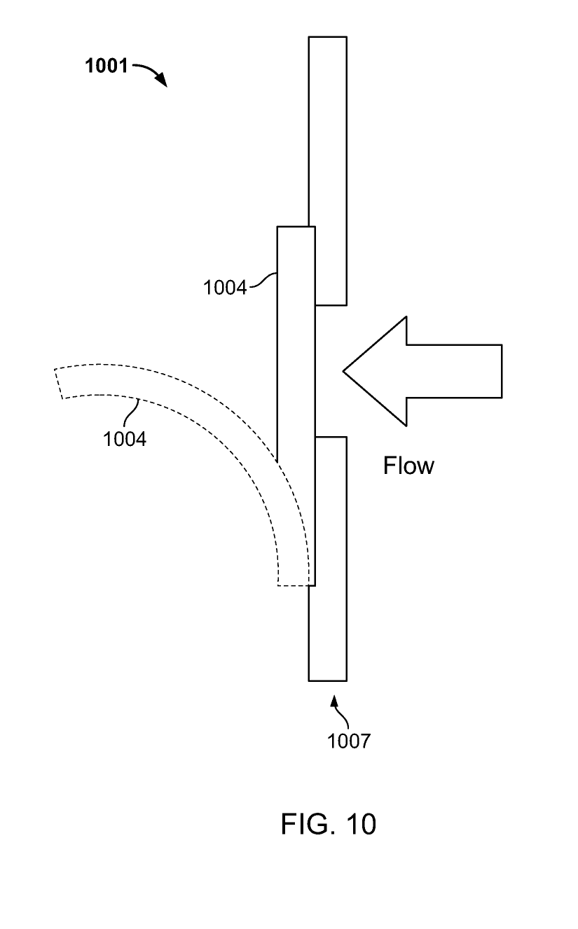

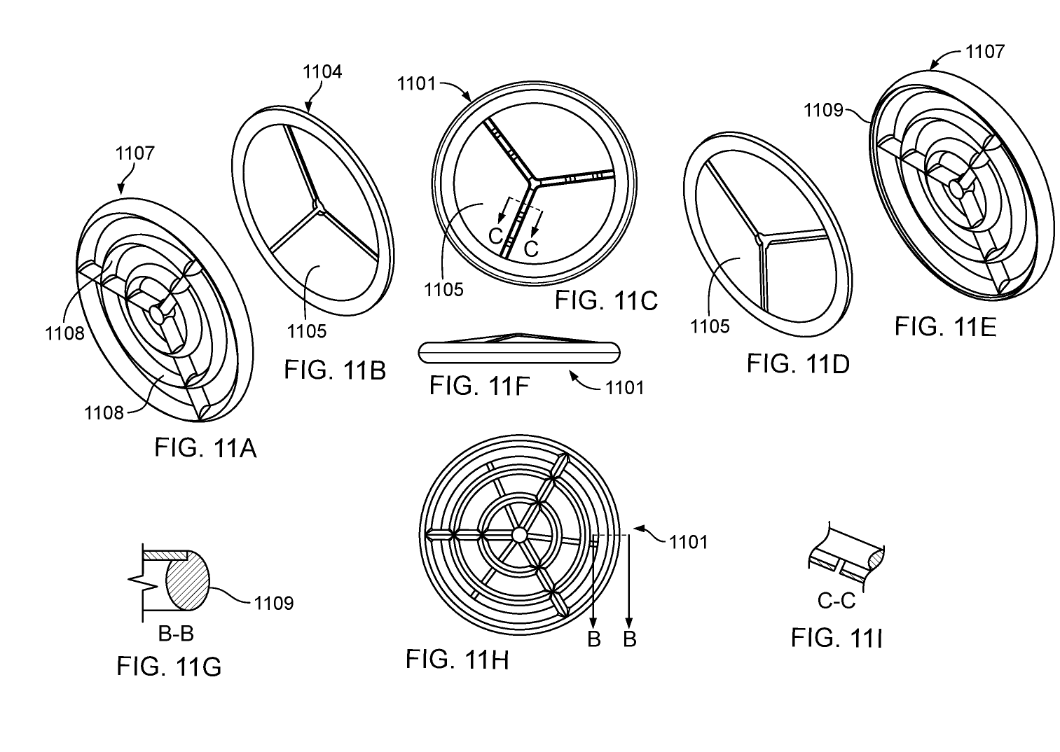

FIG. 10 depicts an implantable transseptal flow control component 1001 according to a ninth embodiment, which includes a shape-stable ring 1007 and a flexible flap 1004 adapted remain in a closed configuration for pressure differentials below a threshold pressure value and to change to an open configuration when a pressure differential exceeds the threshold pressure value.

FIGS. 11A-11I depict an implantable transseptal flow control component 1101 according to a tenth embodiment. As shown, flow control component 1101 includes a compliant disk 1104 having three coapting leaflets 1105 and a shape-stable frame 1107 including multiple passages 1108 there through. In some cases, shape-stable frame 1107 can be a porous structure. An outer ring of shape-stable frame 1107 can include a recess 1109 for receiving compliant disk 1104 to form a secure connection between frame 1107 and disk 1104. When in use, a negative pressure gradient in the direction of compliant disk 1104 can cause leaflets 1105 to deflect allowing fluid flow. Negative pressure gradient in the direction of compliant frame 1107 can force leaflets 1105 to coapt while compliant frame 1107 can prevent leaflets 1105 from inverting. Leaflets 1105 can have shape memory adapted to require a predetermined pressure differential before the leaflets 1105 transition to an open configuration. In some cases, leaflets 1105 can include a shape memory wire (e.g., a nitinol wire) in order to impart shape memory to the leaflets 1105.

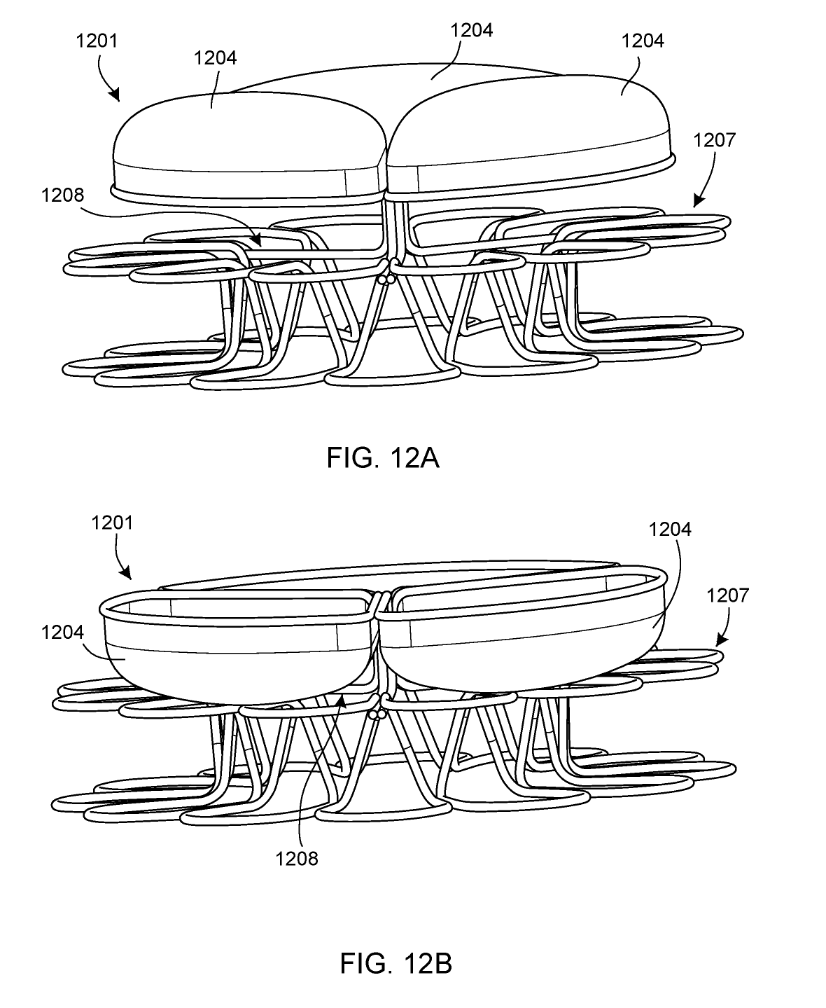

FIGS. 12-12A depict an implantable transseptal flow control component 1201 according to an eleventh embodiment. Flow control component 1201 can include a plurality of lobes 1204. In some cases, flow control component 1204 includes at least 4 lobes. In some cases, flow control component 1204 includes at least 5 lobes, at least 6 lobes, at least 8 lobes, or at least 10 lobes. In some cases, lobes 1204 can be filled with blood via a parachute flow control component. Lobes 1204 are attached to a central location of a frame member 1207 adapted to fit within an opening in a septum between the left atrium and the right atrium. Frame member 1207 can define one or more passages 1208 along its periphery. When the pressure in the right atrium exceeds that of the left atrium, the lobes can deflect towards passages 1208 to close flow control component 1201. When the pressure in the left atrium exceeds that of the right atrium by a predetermined threshold value, lobes 1204 can deflect inward to allow for blood to flow through passages 1208 and around lobes 1205. In some cases, lobes 1204 can include shape memory materials (e.g., nickel-titanium alloy such as nitinol) in order to have flow control component 1201 in a closed configuration when the pressure difference is below a predetermined threshold value.

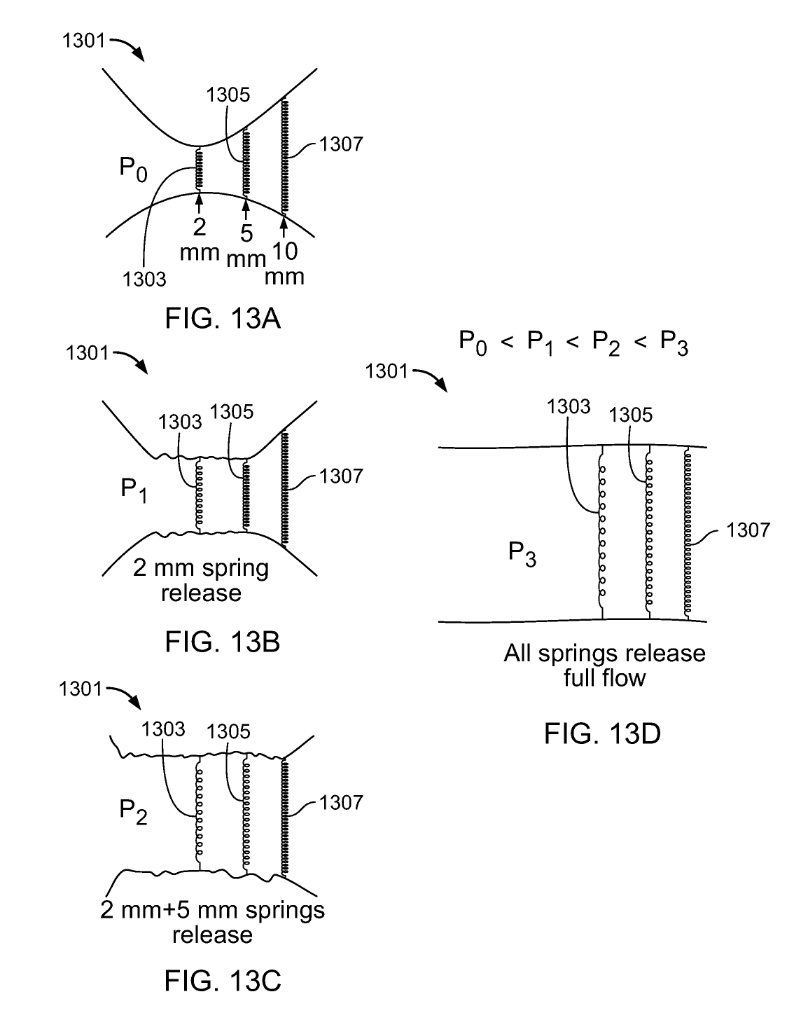

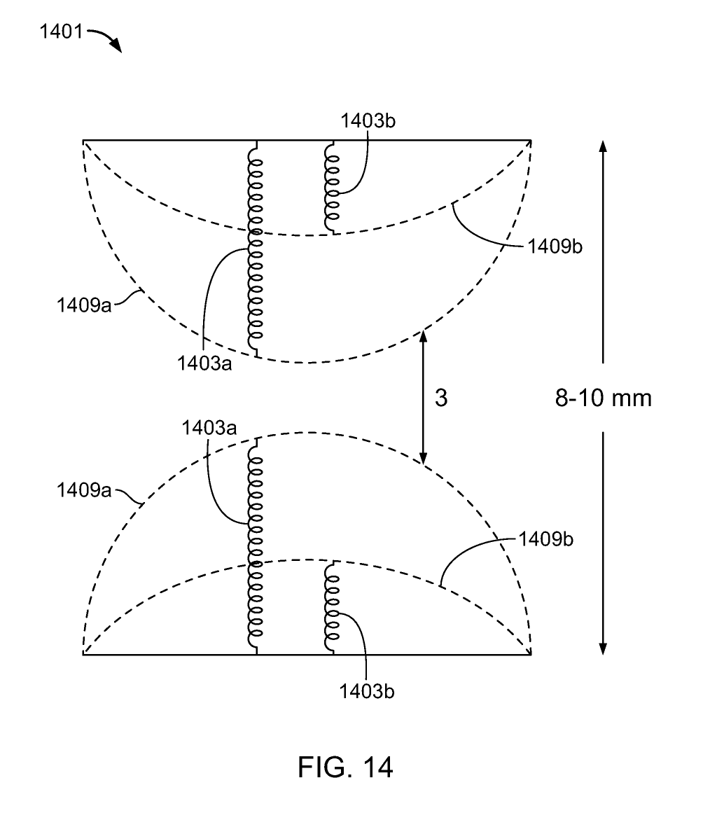

FIG. 13 depicts an implantable transseptal flow control component 1301 according to a twelfth embodiment. FIG. 14 depicts an implantable transseptal flow control component 1401 according to a thirteenth embodiment. Flow control components 1301 and 1401 include a spring-based mechanism. For example, a shape memory material (e.g., stainless steel or a nickel-titanium alloy such as nitinol) can form a frame that opens based on a pressure gradient.

FIGS. 13A-D depicts a flow control component 1301 in four stages based on four different pressures: P₀, P₁, P₂, and P₃. P₀ is less than P₁, which is less than P₂, which is less than P₃. Flow control component 1301 includes three springs 1303, 1305, and 1307. Spring 1303 can be a 0.08 inch (2 mm) spring, spring 1305 can be a 0.2 inch (5 mm) spring, and spring 1307 can be a 0.4 inch (10 mm) spring when the flow control component 1301 is at a pressure of P₀. As shown in FIG. 13, with increasing pressures, springs 1303, 1305, and 1307 can progressively release to allow for the passage to expand to provide an increase in flow. In panel A, for a pressure gradient of P₀, none of the spring mechanisms have actuated. In panel B, the weakest spring 1303 which was maintaining the opening at a 0.08 inch (2 mm) diameter is actuated by pressure P₁, resulting in an opening that is limited by the stronger spring 1305 at the 0.2 inch (5 mm) diameter. In panel C, the pressure P₂ releases the 0.2 inch (5 mm) diameter spring 1305 resulting in a diameter of 0.4 inch (10 mm) limited spring 1307. Finally, as shown in panel D, pressure P3 will release the final spring 1307 allowing for full flow of diameter >0.4 inch (10 mm). In some cases, flow control component 1301 can be arranged such that pressure differential of less than 0.7 kPa (5 mmHg), 1.3 kPa (10 mmHg), or 2 kPa (15 mmHg) will close flow control component 1301.

FIG. 14 depicts a similar arrangement to FIG. 13, but in FIG. 14 springs are compressed with increasing pressures. Flow control component 1401 include one or more springs, such as springs 1403, which is shown in two states, state A 1403a and state B 1403b. At lower pressures, spring 1403 is in state A 1403a, which allows for a reduced width opening or a closure of the opening in flow control component 1401 between channel walls 1409a. When pressure is increased, one or more of the springs will compress to be in state 1403b to allow for an increased opening between channel walls 1409b. Although only one pair of springs (in two states) is shown in FIG. 14, it is contemplated that each side could include multiple springs each having different strengths so as to have the channel expand to different preset diameters with increasing pressures, similar to that described above in reference to FIG. 13. In some cases, flow control component 1401 can be arranged such that pressure differential of less than 1.33 kPa (10 mmHg) will close flow control component 1401. In some cases, springs 1403 can be circumferential for a round flow lumen. In some cases, springs can be linear springs. In some cases, linear springs can be included in the bill design previously disclosed herein.

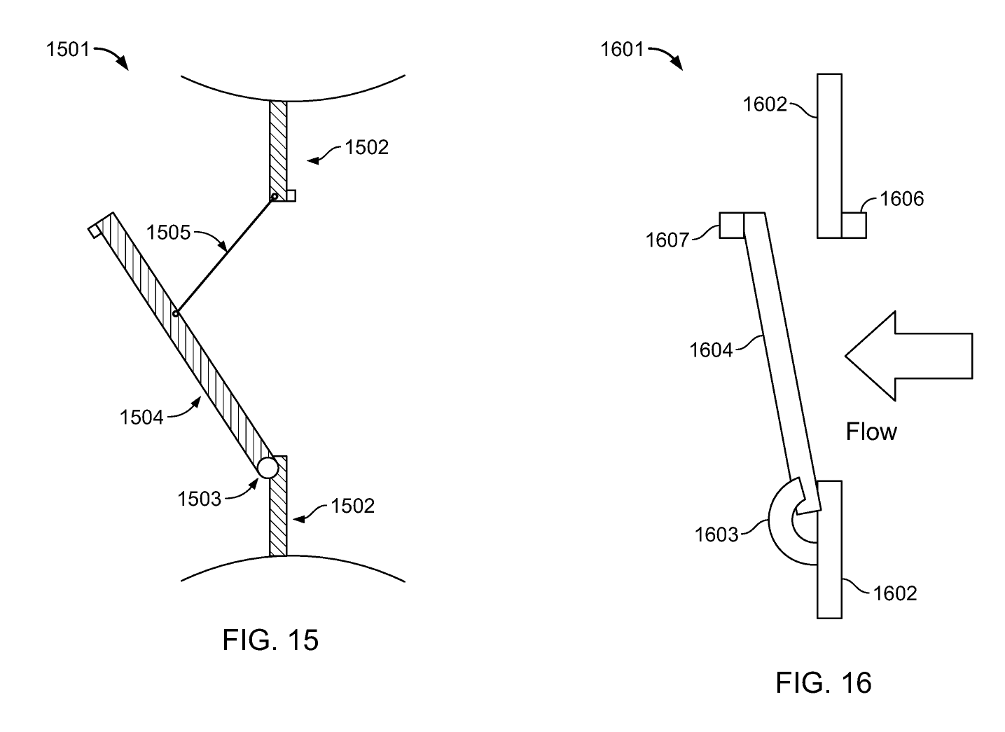

FIG. 15 depicts an implantable transseptal flow control component 1501 according to a fourteenth embodiment. As shown, flow control component 1501 includes a frame 1502 and a hinged-door 1504 connected via hinge 1503, a limiting cord 1505, and a set of magnets 1506 and 1507 positioned on hinged door 1504 and frame 1502 opposite hinge 1503. For flow control component 1501, the strength of the magnetic pull can balance the pressure force from the blood such that the hinged door 1504 only moves to an open configuration upon a pressure difference between the left atrium and right atrium exceeding a predetermined threshold value. In some cases, a spring can be provided to bias the hinged door towards a closed configuration.

FIG. 16 depicts an implantable transseptal flow control component 1601 according to a fifteenth embodiment. Flow control component 1601 also includes a frame 1602 and a spring loaded hinged door 1604 connected via a spring loaded hinge 1603 and a set of magnets 1606 and 1607. Spring loaded hinge can bias the hinged door towards a closed configuration and have the opening grow large with greater pressure differentials.

Magnets, springs, and/or limiting cords such as shown in FIGS. 15 and 16 can be applied to each of the other embodiments discussed herein.

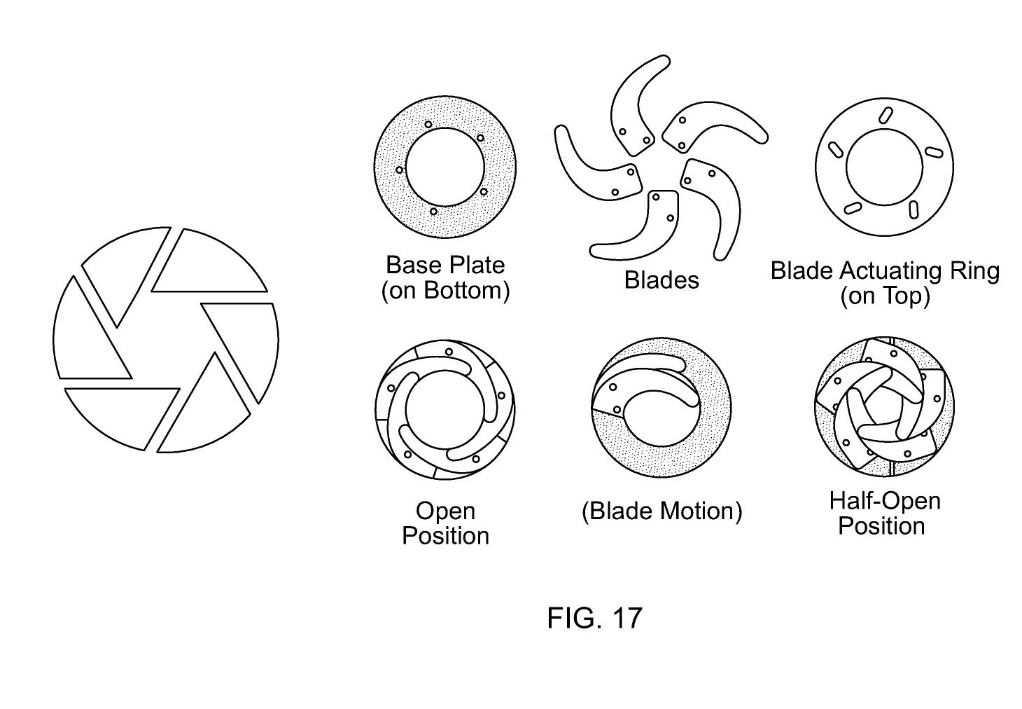

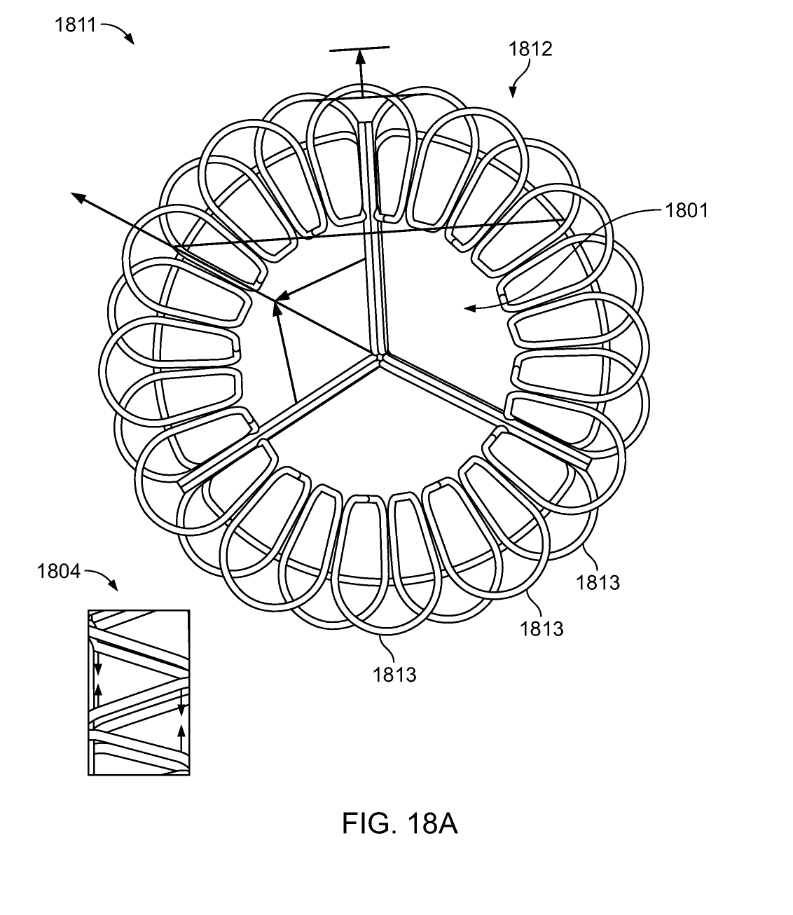

In some cases, a flow control component provided herein can be a metallic iris, such as the iris designs shown in FIG. 17. A metallic iris can be can be activated by electrical energy from a supply source. An electrical energy supply source can be internal or external to the body. An external source—for example, a doctor or nurse can use an RF transmitter externally to activate the flow control component which has an RF receiver. An internal source—in some cases, a pacemaker or ICD battery can be implanted and used to activate a flow control component or using the existing battery of a pacemaker or ICD already implanted in the diseased patient. The opening of the iris can be modulated in some cases using imaging modalities, such as fluoro or TEE/ICE, can be used to determine an appropriate opening of a hole in the septum between the left atrium and the right atrium. Alternatively, the iris could have a pressure sensor on the LA side or both on the LA and RA side and then based on a feedback loop system be opened using the internal energy source once a pre-determined and calibrated threshold has been achieved.

The iris can be mounted on a shape-stable ring such as in FIG. 9A i.e. 902. The default state of the iris can be in a closed position.

In some cases, systems provided herein can include controllers adapted to control the opening of a flow control component provided herein. A controller can be implanted or external. In some cases, the controller can activate control based on an algorithm within electronics such as a pacemakers or ICD type device to open a specific amount at specific times of the day (such as during sleep) or during specific activities (such as during exercise). Alternatively, the opening of the device can be based on internal feedback from one or more pressure sensors placed just in the left atrium or in both the left and right atriums. In some cases, pressure sensors can be incorporated into flow control components provided herein or mounted separately in the body, such as on the left atrial appendage closure frame placed in the left atrial appendage.

In some cases, methods and systems provided herein can monitor the number of times or rate of activation of a flow control component provided herein and transmit that value through RF signals to an external display unit. In some cases, a doctor or nurse could find a rate, time, and/or change in activation useful for evaluating the progression of heart failure. In some cases, flow control components provided herein can be monitored for appropriate operation by detecting a sound of the flow control component opening and closing similar to standard heart sounds. For example, flow control components provided herein can be designed to create a sound undetectable to a human ear, but detectable by an electronic sensor. In some cases, flow control components provided herein can include piezoresistive or piezoelectric elements that are activated by the open-close cycle and transmit this information to an external device through RF or to an internal device such as the pacemaker or ICD or standalone implantable controller. In some cases, an internal implantable device can be included in systems provided herein or used in methods provided herein to monitor flow control components provided herein. For example, an internal implantable device for monitoring flow control components provided herein can be similar to a low voltage pacing system. In some cases, a monitoring system can be incorporated into another implanted device, such as a pacemaker, which may be able to allow for continuous monitoring and the upload of data via telemetry.

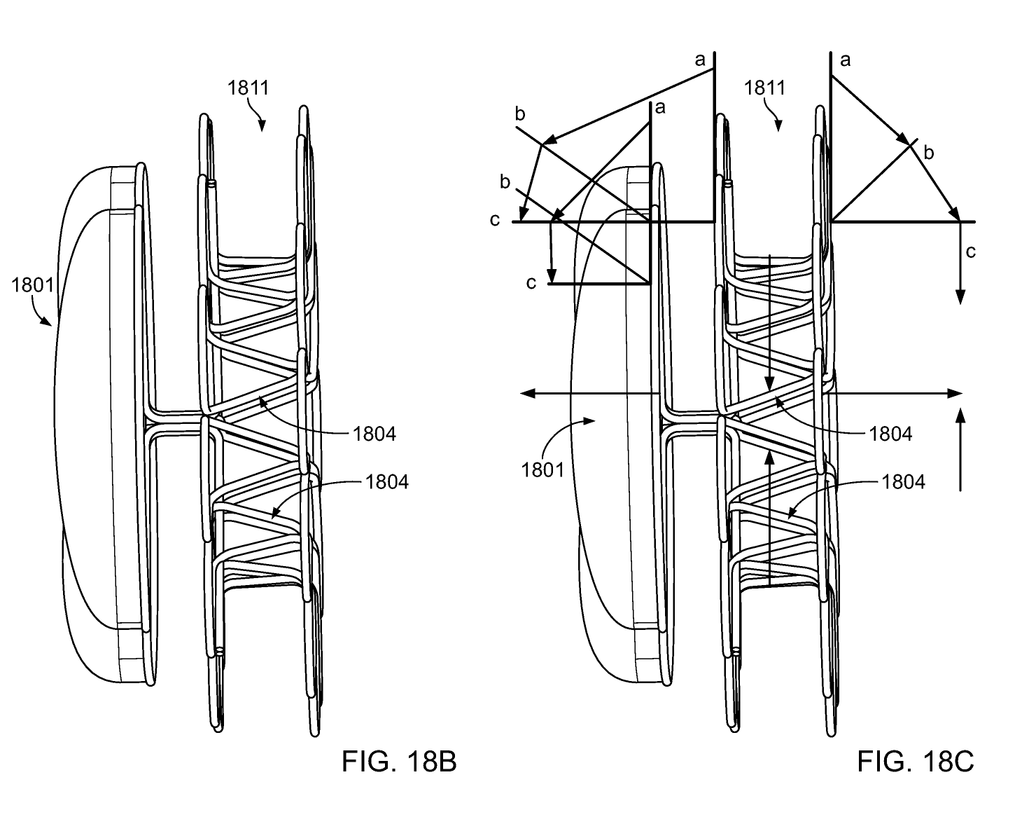

FIGS. 18A-18 C depict a support frame 1811 for a collapsible shape-stable member, which can be used with implantable transseptal flow control components provided herein. FIGS. 18A-18C depict flow control component 1801. Frame 1811 includes wire segments that form a ring 1812, which can be used to stretch a non-elastic material to form a shape-stable member in the embodiments discussed above in regards to FIGS. 3A-16. Frame 1811 can collapse when sheared in directions parallel with the axis of ring 1812. The wire segments making up ring 1812 includes a series of loops 1813 and V-shaped elements 1814, which are capable of transformation into a generally tubular, constrainable shape. In some cases, frame 1811 can include a shape memory material (e.g., a nickel-titanium alloy such as Nitinol). In some cases, a phase transition of a shape memory material can be used to control the expansion and retraction of the frame from a collapsed configuration to a shape-stable expanded configuration.

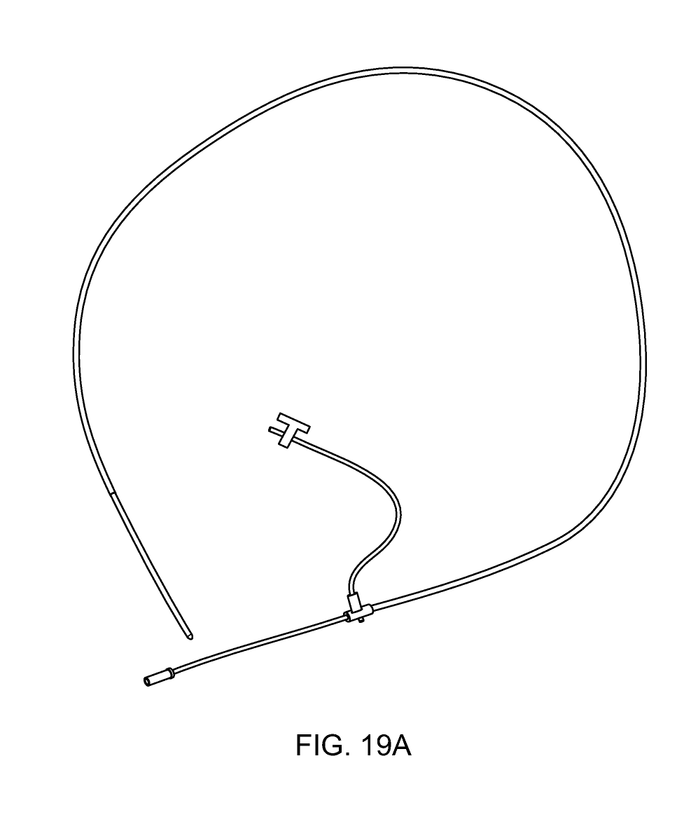

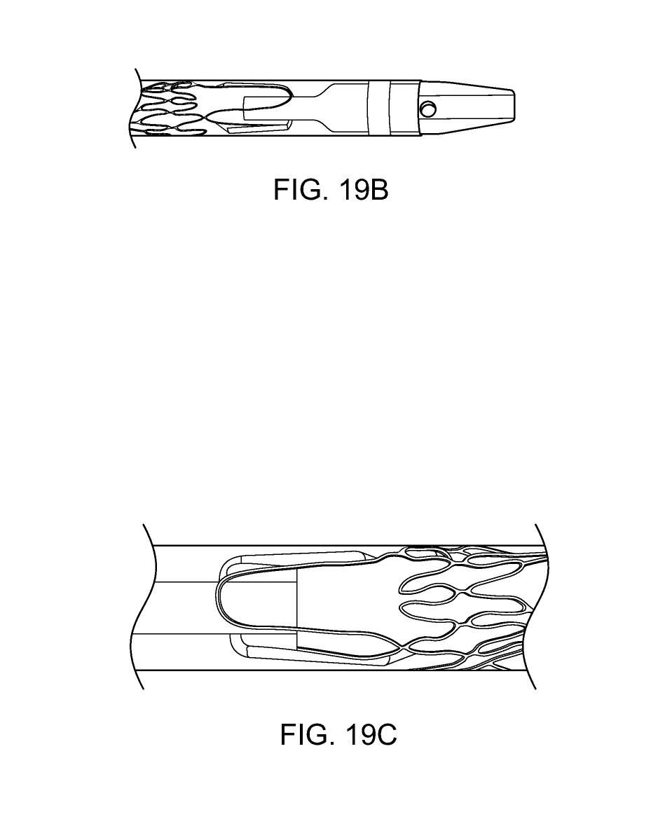

For example, with Nitinol, cooling the structure to its Martensitic state, the structure can be significantly manipulated without damage to the structure. As long as the low martensitic temperature is maintained the structure will remain very ductile and retain its manipulated shape until warmed. The first step to the transition to a constrainable tubular form is to chill the structure to its martensitic state and maintain the cool environment. Referring to FIGS. 18B and 18C, the structure can be manually manipulated to generally orientate the loops 1813 into an advantageous position for final constrainment. All of the loops 1813 can be reoriented, e.g., partially tipped or bent, into the desired direction from the depicted plane a to somewhere beyond the depicted plane b, towards the plane c. Loops can be reoriented using cool metal tools while maintaining a cool environment (e.g., cold air or submerged in very cold solution). Once the loops have achieved a general constrainable orientation (sufficient bias to guide all loops in the desired direction when force constrained in an iris) as shown in a similar sample structure, the device can be placed into a pre-cooled constrainment iris tool. The structure can be constrained into a loadable tubular state and is ready to be loaded onto the delivery catheter. As long as the device is kept below its austenitic start temperature, it will remain in the constrained tubular state. Once loaded into the delivery catheter, such as shown in FIGS. 19A-19C, in a similar manner as the like device that is depicted, the device will remain in that orientation until deployed into its final physiological deployment location. When the outer sheath is retracted in the body, the device will warm from the heat of the blood. This will phase transition the metal through its austenitic range to its finish temperature. The device will return to its stress relieved original shape. The catheter is withdrawn and the device will function as designed.

In some cases, embodiments of the flow control components discussed herein can be configured such that blood does not stagnate at a location proximate to a left atrium flow control component side when the pressure differential is below a second predetermined threshold pressure value. In some cases, embodiments of the flow control component discussed herein can be configured such a periodic opening of the flow control component during each cardiac cycle is less than 100 ml/minute to prevent stagnation.

A number of embodiments of the invention have been described. Nevertheless, it will be understood that various modifications may be made without departing from the spirit and scope of the invention. Accordingly, other embodiments are within the scope of the following claims.

What is claimed is:

1. An implantable transseptal flow control component comprising at least a first member, the flow control component being adapted to be implanted in an opening in a septal wall between a left atrium and a right atrium, the flow control component being adapted to prevent blood from flowing through the opening when in a closed configuration, the flow control component being adapted to remain in a closed configuration when a pressure differential between the left atrium and the right atrium is less than a first non-zero predetermined threshold pressure value and transition into an open configuration when the pressure differential exceeds the first non-zero predetermined threshold pressure value, wherein the closed configuration is configured such that blood does not stagnate at a location proximate to a left atrium flow control component side when the pressure differential is below a second predetermined threshold pressure value.

2. The flow control component of claim 1, wherein the second predetermined threshold pressure value is equal to the first non-zero predetermined threshold pressure value.

3. The flow control component of claim 1, where the second predetermined threshold pressure value is less than the first non-zero predetermined threshold pressure value.

4. The flow control component of claim 1, wherein the open configuration defines a passage through the flow control component that increases with an increasing pressure differential after the pressure differential exceeds the first non-zero predetermined threshold pressure value.

5. The flow control component of claim 1, wherein the first member is adapted to flex in response to pressure differential.

6. The flow control component of claim 5, wherein the first member defines a collapsed passage there through when the pressure differential is less than the second predetermined threshold pressure value.

7. The flow control component of claim 5, further comprising a second member, the second member configured to form a shape-stable support structure when the flow control component is implanted.

8. The flow control component of claim 7, wherein the second member defines a passage there through and the first member overlies and seals the passage when the pressure differential is below the second predetermined threshold pressure value.

9. The flow control component of claim 7, wherein the first member has a semi-circular shape.

10. The flow control component of claim 7, wherein the first member defines at least one passage there through.

11. The flow control component of claim 10, wherein the first and second members are both disk shaped and connected along a periphery of the disks or at a central location of each disk.

12. The flow control component of claim 1, wherein the first member comprises a shape memory metal.

13. The flow control component of claim 1, wherein the first member forms at least one lobe structure.

14. The flow control component of claim 1, wherein the flow control component comprises a spring, a magnet, or a combination thereof.

15. The flow control component of claim 1, further comprising a controller adapted to detect the pressure differential and control the opening and closing of the flow control component based on the detected pressure differential.

16. An implantable transseptal flow control component comprising a shape-stable member and a compliant member, the flow control component being adapted to be implanted in an opening in a septal wall between a left atrium and a right atrium, at least one of the shape-stable member and the compliant member defining a passage there through, the shape-stable member and the compliant member being attached at at least one location and overlying each other to seal off any passages through the flow control component when the flow control component is in a closed configuration to prevent blood from flowing through the opening, the flow control component being adapted to remain in a closed configuration when a pressure differential between the left atrium and the right atrium is less than a first non-zero predetermined threshold pressure value and transition into an open configuration when the pressure differential exceeds the first non-zero predetermined threshold pressure value.

17. The flow control component of claim 16, wherein the shape-stable member is adapted to be collapsed for insertion and expanded for placement within the opening, the shape-stable member being compliant in the expanded configuration.

18. The flow control component of claim 16, wherein shape-stable member and the compliant member are each disk shaped and attached and sealed together at a central location or along a periphery of at least one disk.

19. The flow control component of claim 16, further comprising a shape memory wire in the compliant member.

20. An implantable transseptal flow control component comprising a compliant member defining a collapsed passage there through, the compliant member being adapted to be implanted in an opening in a septal wall between a left atrium and a right atrium, the collapsed passage being adapted to remain in a closed configuration when a pressure differential between the left atrium and the right atrium is less than a first non-zero predetermined threshold pressure value and transition into an open configuration when the pressure differential exceeds the first non-zero predetermined threshold pressure value.
